(19) United States
(12) Patent Application Publication
(10) Pub. No.: US 2023/0249442 A1
(43) Pub. Date: Aug. 10, 2023

(54) Apparatus having a housing made of an at least three-layer material for releasing a volatile substance into the ambient air

(71) Applicant: SWIF GmbH South West International Fragrance, Kaiserslautern (DE)

(72) Inventor: Jörn Karg, Kaiserslautern (DE)

(21) Appl. No.: 18/105,956

(22) Filed: Feb. 6, 2023

(30) Foreign Application Priority Data
Feb. 7, 2022 (DE) 102022201257.7

(51) Int. Cl. B32B 27/08; B32B 27/10; B32B 27/32; B32B 27/34; B32B 27/36; B32B 27/30; B32B 7/12; B32B 15/20; B32B 15/085; B32B 3/26; A61L 9/12; A61L 9/04

(52) U.S. Cl. CPC B32B 27/08; A61L 9/12; A61L 9/042; A61L 9/046; B32B 3/266; B32B 7/12; B32B 15/20; B32B 15/085; B32B 27/10; B32B 27/32; B32B 27/34; B32B 27/36; B32B 27/306; B32B 2255/10; B32B 2255/12; B32B 2255/26; B32B 2307/412; B32B 2307/516; B32B 2307/518; B32B 2307/7242; B32B 2307/7265; B32B 2439/40

(57) ABSTRACT

An apparatus for releasing a volatile substance into the air that includes a housing and an insert, the housing contains an at least three-layer first material; wherein a first layer of the at least three-layer first material contains or comprises paper or cardboard or paperboard, a second layer of the at least three-layer first material contains or comprises polyethylene, a third layer of the at least three-layer first material contains or comprises oriented polypropylene, the third layer has a barrier function against permeation of the volatile substance, and optionally the apparatus has at least one further barrier layer having a barrier function against permeation of the volatile substance, the insert is arranged inside the housing and made of a second material, the second material is provided or impregnated with the volatile substance.

### PRIORITY

This application claims priority to DE 10 2022 201 257.7 filed on Feb. 07, 2022, the entirety of which is hereby incorporated by reference herein for all purposes.

### FIELD

These teachings relate to an apparatus for releasing a volatile substance, in particular a fragrance or an insecticide, into the ambient air, with a housing having an at least three-layer first material and wherein a first layer contains or comprises paper or cardboard or paperboard, a second layer contains or comprises polyethylene (PE), and a third layer contains or comprises oriented polypropylene (e.g. BOPP).

### BACKGROUND

Similar apparatus are known, for example, from US 4 258 874 A, US 4 166 565 A, and US 4 279 373 A, and have a housing embodied, for example, from paper or cardboard. A releasing device is typically arranged inside the housing and can release a volatile substance, e.g., a fragrance, during use of the apparatus. It has proven disadvantageous with these apparatuses that the volatile substance can diffuse through the housing made of paper or paperboard even prior to the apparatus actually being used. Because of this, only a reduced quantity of the volatile substance is available to the user during the usage interval. A further drawback is that residues that are not biodegradable typically remain in the housing after the apparatus has been used. This complicates disposal and recycling of such apparatus.

A further apparatus for releasing a fragrance is known from US 4 712 737 A. This apparatus comprises a housing made of coated cardboard and a releasing device arranged inside the housing that can be formed, for instance, from a gel system, woven material, or nonwoven material. In this apparatus, as well, residues that are not biodegradable may remain in the housing after use.

German patent application no. 10 2020 120 951.7 discloses a device for releasing a volatile substance, in particular a fragrance or insecticide, into the ambient air.

### SUMMARY

An object of the present teachings is to facilitate the disposal and/or recycling of an apparatus for releasing a volatile substance into the ambient air.

For attaining the object, an apparatus for releasing a volatile substance, in particular a fragrance or an insecticide, into the ambient air is disclosed that includes a a) housing (10) that contains an at least three-layer first material and wherein (i) a first layer of the at least three-layer first material contains or comprises paper or cardboard or paperboard, and (ii) a second layer of the at least three-layer first material contains or comprises polyethylene (PE), and (iii)

a third layer of the at least three-layer first material contains or comprises oriented polypropylene (e.g. BOPP) and at least this third layer has a barrier function against permeation of the volatile substance, and optionally has one or a plurality of further barrier strata having a barrier function against permeation of the volatile substance, and b) an insert (20) arranged inside the housing (10) and made of a second material, wherein the second material is provided, in particular impregnated, with the volatile substance, wherein the second material is one of the following materials or has one or a plurality of the specified materials: a plant-based fiber material or sucrose or starch or a mineral material.

Such apparatus can also be called air fresheners or insecticides or insect repellants.

The inventive apparatus comprises a housing made of a first material. This first material has a first layer containing or comprising paper, cardboard, or paperboard, a second layer containing or comprising polyethylene (PE), and a third layer containing or comprising oriented polypropylene (e.g., BOPP). A further component of the apparatus is an insert made of a second material that differs from the first material. Thus, the first and second materials can be selected specifically for their individual function. This is because while the first material of the housing must provide a barrier function for the volatile substance prior to the apparatus being used, the second material should have the best possible properties for releasing the volatile substance. The second material is a plant-based fiber or sucrose or starch or a mineral material. The second material is provided, impregnated, with the volatile substance, so that the latter can be released from the housing into the ambient air. The insert of the apparatus should also be simple to dispose of or recycle after use. It is possible to dispose of the housing and the insert together, i.e., without having to separate them, e.g. with waste paper or packaging waste (“yellow bag”).

According to the teachings herein, the first layer (typically “outer layer”) of the first material contains or comprises the paper, cardboard, or paperboard, in particular cardboard, i.e. preferably a specific plant-based fiber material. An “outer layer” is preferably a layer that, when the finished apparatus is viewed, is at least partially visible without having to disassemble the finished apparatus. If, without having to disassemble the finished apparatus, a plurality of strata are at least partially visible when the finished apparatus is viewed, the apparatus preferably comprises a plurality of “outer strata.” An “outer layer” can preferably be construed to mean a layer that spatially faces away from the insert. The first layer of the first material, which contains or comprises paper, cardboard, or paperboard, is preferably an “outer layer.”

According to the teachings herei, the second layer of the first material, which contains or comprises polyethylene (PE), acts as an adhesion agent between the first layer of the first material and the third layer of the first material. Preferred polyethylenes include low-density polyethylene (LDPE/PE-LD), linear low density polyethylene (LLDPE/PE-LLD), and/or high density polyethylene (HDPE/PE-HD). “LDPE” means polyethylene of low density, which can have a density in the range of, e.g. 0.86 to 0.93 g/cm³, and is distinguished by a high degree of branching by the molecules. “HDPE” means polyethylene of high density, which has only slight branching of the molecule chain, wherein the density can be in the range of, e.g., 0.94 to 0.97 g/cm³.

According to the teachings herein, the third layer (typically “inner layer” of the first material, which contains or includes oriented polypropylene (e.g. BOPP), acts as a barrier layer. The polypropylene can extend, e.g., in a transverse or longitudinal direction (monoaxial = OPP) or even in both directions (biaxial = BOPP). This layer has excellent barrier properties against, for example, odors, aromas, gases, and water vapor. The third layer of the first material is preferably water-repellant and/or, at least in part, acrylic-coated. Furthermore, this barrier layer has a barrier function against permeation of the volatile substance. Such a barrier substance is understood to be a layer that has low permeability for the volatile substance. The barrier layer can prevent the volatile substance from escaping from the housing prior to the apparatus being activated. Due to the barrier layer, high enough partial pressure of the volatile substance can be maintained in the interior of the housing for further diffusing of the volatile substance out of the insert to be prevented. An “inner layer” is preferably a layer that, when the finished apparatus is viewed, is at least partially not visible. If, without having to disassemble the finished apparatus, a plurality of strata are at least in part not visible when viewing the finished apparatus, the apparatus preferably comprises a plurality of “inner strata.” An “inner layer” can preferably be construed to mean a layer that spatially faces the insert. The third layer of the first material, which contains or comprises oriented polypropylene (e.g., BOPP), is preferably an “inner layer.”

Suitable paper, cardboard, paperboard, polyethylene (PE), and oriented polypropylene (e.g., BOPP) are fundamentally known and commercially available to the person skilled in the art.

The (i) first layer of the at least three-layer first material, the (ii) second layer of the at least three-layer first material, and/or the (iii) third layer of the at least three-layer first material may each contain, if necessary, independently of one another, one or more additives selected from the group comprising antistatic agents, antioxidants, antiblocking agents, antifogging agents, antimicrobial substances, dyes, pigments, stabilizers, preferably heat stabilizers, process stabilizers, processing aids, flame retardants, nucleation agents, crystallization agents, preferably crystal nucleating agents, slip agents, optical brighteners, flexibilizing agents, sealing agents, softeners, silanes, spacers, filling agents, peel additives, waxes, wetting agents, surface-active compounds, preferably surfactants, and dispersing agents.

The (i) first layer of the at least three-layer first material, the (ii) second layer of the at least three-layer first material, and/or the (iii) third layer of the at least three-layer first material may each contain, if necessary, independently of one another, 0.01-20% by weight, preferably 0.1-10% by weight, in each case relative to the total weight of an individual layer, of at least one of the aforesaid additives.

In addition to the third layer of the first material, which contains or comprises oriented polypropylene (e.g. BOPP), the housing can optionally have a further barrier layer with a barrier function against permeation of the volatile substance.

The further barrier layer preferably comprises polyamide, polyester, aluminum, ethylene vinyl alcohol, silicon dioxide, and/or polyvinyl alcohol. These materials have good barrier properties, in particular low permeability for volatile substances, in particular fragrances and insecticides. An embodiment with polyvinyl alcohol offers the advantage that the further barrier layer is water soluble and is therefore simple to dispose of. Overall simplified recycling of the apparatus is thus made possible.

The first material can also comprise a plurality of further barrier strata, preferably two, three, four, or five. Each of the individual further barrier strata can comprise polyamide, polyester, aluminum, ethylene vinyl alcohol, silicon dioxide, and/or polyvinyl alcohol. For example, it can be provided that the further barrier strata have different permeabilities for different volatile substances. In this way a housing can be obtained that has a barrier function for a plurality of volatile substances, for example, for a plurality of substances in a mixture of substances, e.g. a fragrance mixture.

The further barrier layer is preferably arranged between two strata of the first material, preferably between the first layer of the first material and the second layer of the first material or between the second layer of the first material and the third layer of the first material. In this way it is possible to obtain a compact design of the housing. Furthermore, the further barrier layer can be protected from environmental influences by the two strata of the first material. When using a further barrier layer made of polyvinyl alcohol, the former can be protected from the effects of water, for example, by the two strata of the first material and thus undesired damage to the further barrier layer can be prevented. Optionally, one additional further layer, for example a protective layer and/or a sealing layer, can be arranged between the further barrier layer and each of the two strata of the first material.

Alternatively, it is preferred when the further barrier layer is embodied as a coating of a layer of the first material, in particular as a coating on an inner side of the housing, e.g., the third layer of the first material.

In one preferred embodiment, the first material contains exactly three strata, specifically (i) a first layer that contains or comprises paper, cardboard, or paperboard, in particular cardboard, (ii) a second layer that contains or comprises polyethylene (PE), and (iii) a third layer that contains or comprises oriented polypropylene (e.g. BOPP). Such a construction corresponds to the arrangement of “paper, cardboard, or paperboard as outer layer, polyethylene as adhesion agent, oriented polypropylene as inner layer.”

One advantageous embodiment of the teachings herein provides that the housing has a stand surface on which the housing can stand on a subsurface, wherein the stand surface is water repellant. For example, the housing can have a water-repellant coating in the region of the stand surface. The coating can comprise a nanomaterial.

One advantageous embodiment of the teachings herein provides that the plant-based fiber material is paper or cardboard or paperboard, for example, mechanical pulp board or wood pulp.

One advantageous embodiment of the teachings herein provides that the plant-based material is mechanical pulp, in particular ground wood pulp, or pulp or cotton fiber or grass fiber.

One advantageous embodiment of the teachings herein provides that the mineral material is rock flour or bentonite or silica gel.

One advantageous embodiment of the teachings herein provides that the insert has a plurality of plies of the second material connected to one another, in particular by an adhesive, in particular a plurality of plies of mechanical pulp board. Because the insert is formed from a plurality of plies of the plant-based fiber material, the weight of the insert, and thus also the weight of the entire apparatus, can be adjusted. It has been found that the users of such apparatus find it more valuable when the apparatus have a certain weight - that is, are not too light in weight. Therefore, a prespecified weight of the apparatus can be set by selecting the number of plies, for example four or five or six or seven or eight. In addition, as a result of the increased weight of the insert, there is the advantage that the volatile substance, in particular the fragrance or the insecticide, is distributed over a larger mass of the insert. Because of this, the volatile substance is diluted such that no unpleasantly strong odor is perceivable in the immediate vicinity of the housing of the apparatus. This circumstance can also contribute to the apparatus being perceived by the user as more valuable. As an alternative to the plies being connected using an adhesive, the plies can be connected using one or a plurality of nails, clamps, eyelets, bolts, or strips, for example a strip made of metal, wood, a polymer, glass, or stone.

If the insert has a plurality of plies of mechanical pulp board, it is advantageous when the mechanical pulp board comprises a layer of ground wood pulp arranged between two cover strata. The cover strata preferably each comprise pulp or paper, particularly preferably pulp and paper. The thickness of the mechanical pulp board is preferably in the range of 0.5 mm to 2.5 mm, for example 0.8 mm, 0.9 mm, 1.2 mm, 1.4 mm, 1.6 mm, 1.8 mm, 2.0 mm, or 2.4 mm.

According to one advantageous embodiment it is provided that the plys each have an area density in the range of 350 g/m² to 950 g/m², preferably 450 g/m² to 600 g/m², or 600 g/m² to 800 g/m². Plys having such an area density have proven to be particularly good for adjusting the weight of the insert.

Alternatively, it is advantageous when each of the plys has a thickness in the range of 0.2 mm to 2.5 mm and an area density in the range of 350 g/m² to 950 g/m², preferably:
a thickness in the range of 0.8 mm to 1.0 mm and an area density in the range of 350 g/m² to 400 g/m², or,
a thickness in the range of 1.1 mm to 1.3 mm and an area density in the range of 450 g/m² to 550 g/m², or,
a thickness in the range of 1.3 mm to 1.5 mm and an area density in the range of 490 g/m² to 570 g/m², or,
a thickness in the range of 1.5 mm to 1.7 mm and an area density in the range of 580 g/m² to 660 g/m², or,
a thickness in the range of 1.7 mm to 1.9 mm and an area density in the range of 630 g/m² to 750 g/m², or,
a thickness in the range of 1.9 mm to 2.1 mm and an area density in the range of 700 g/m² to 850 g/m², or,
a thickness in the range of 2.3 mm to 2.5 mm and an area density in the range of 800 g/m² to 1000 g/m².

Plies having such a combination of thickness and area density have proven to be particularly good for adjusting the weight of the insert.

According to one advantageous embodiment of the teachings herein, it is provided that the adhesive is biodegradable. By using a biodegradable adhesive, it is possible to provide an insert that is simpler to dispose of, in particular that is simpler to recycle or subject to industrial composting. It is particularly preferred that the adhesive conforms to European Standard EN 13432 and is “completely biodegradable” according thereto. Alternatively, an adhesive that is not biodegradable can be used.

One advantageous embodiment of the teachings herein provides that the insert has at least a first ply, embodied in particular with the full surface area, and at least one second ply having at least one recess, in particular a punched hole. The punched hole can form a receiving element for the volatile material. In this way it is possible to add the volatile material, for example in liquid form, to the receiving element formed by the recess. The volatile material can collect in the insert starting from the recess. The at least one recess can be embodied longitudinally, for example as a groove.

In this context, an embodiment in which the second ply forms an outer surface of the insert has proven advantageous.

One advantageous embodiment provides that the insert has a plurality of second plies, wherein the recesses of the plurality of second plies are arranged congruently above one another.

One advantageous embodiment of the teachings herein provides that a time-temperature indicator is arranged on an outer side of the housing. The end of the useful life of the apparatus can be displayed by the time-temperature indicator. In this way the apparatus can display to a user whether the apparatus can still be used or can be disposed of or provided for recycling. The time-temperature indicator preferably comprises heat and UV light-sensitive pigments.

These pigments can be activated by a manufacturer of the apparatus at the end of the production process by irradiating with UV light, wherein the UV dosage used here conforms to the shelf life. It is particularly preferred that the heat and UV light-sensitive pigments are covered by a film that is impermeable to UV light so that later manipulation using UV light is not possible.

One advantageous embodiment of the teachings herein provides that the housing has a first housing part and a second housing part, wherein the first housing part and/or the second housing part comprises a receiving element for the insert, wherein the first housing part comprises one or a plurality of openings, in particular through-holes, through which the volatile substance can be released into the ambient air, and the second housing part can be displaced from a closed position, in which it covers the openings, in particular through-holes, of the first housing part to an open position, in which the openings, in particular through-holes, of the first housing part are exposed for releasing the volatile substance. In one such embodiment, the release of the volatile substance can be activated by displacing the two housing parts relative to one another. In the closed position, the second, in particular outer, housing part covers the through-holes in the first, in particular inner, housing part. Since the first material of the inner and outer housing part limit, in particular prevent, escape of the volatile substance, the partial pressure of the volatile substance within the housing remains constant at a level preventing escape of the volatile substance from the insert. When the apparatus is activated by displacing the housing parts, the one or plurality of through-holes are exposed. Consequently, ambient air can circulate through the through-holes and the volatile substance can be released into the ambient air.

The first and second housing parts preferably have a basic outline of identical shape, for example round, triangular, rectangular, in particular square, pentagonal, hexagonal, or octagonal.

A locking element that prevents complete separation of the two housing parts from one another is preferably arranged on the first housing part and/or second housing part. Because of this, undesired exposure of the insert, in particular by children, can be prevented. In this way it is possible to increase child safety using such an embodiment.

One alternative advantageous embodiment of the teachings herein provides that the housing has a first housing part, a second housing part, and a third housing part, wherein the first housing part and/or the second housing part comprises a receiving element for the insert, wherein the third housing part has one or a plurality of through-holes through which the volatile substance can be released into the ambient air, wherein the first housing part and/or the second housing part can be displaced from a closed position, in which the first housing part and/or the second housing part covers the through-holes of the third housing part, to an open position, in which the through-holes of the third housing part are exposed for releasing the volatile substance. In such an embodiment, the volatile substance can be released by displacing the first and/or second housing part relative to one another to the third housing part. In the closed position, the first and/or second housing part covers the through-holes in the third housing part, which is arranged in the closed position preferably within the first and/or second housing part. Since the first material of the housing limit, in particular prevent, escape of the volatile substance, the partial pressure of the volatile substance within the housing remains constant at a level preventing escape of the volatile substance from the insert. When the apparatus is activated by displacing the first and/or second housing part relative to the third housing part, the one or plurality of through-holes are exposed. Consequently, ambient air can circulate through the through-holes and the volatile substance can be released into the ambient air.

In one such embodiment, the first housing part and the second housing part can be embodied identical. The third housing part can have the shape of a tube, wherein the basic outline of the tube is of the identical shape as a basic outline of the first and/or second housing part, in particular round, triangular, rectangular, in particular square, pentagonal, hexagonal, or octagonal.

A first locking element is preferably arranged on the third housing part and cooperates with a further first locking element on the first housing part and a second locking element is arranged on the third housing part and cooperates with a further second locking element on the second housing part. Complete separation of the first and second housing parts from the third housing part can be prevented using these locking elements. Because of this, undesired exposure of the insert, in particular by children, can be prevented. Thus, child safety can be increased using such an embodiment.

According to one advantageous embodiment, a gap between the first housing part and the second housing part in the closed position is covered by a removable barrier layer having or embodied by a barrier material having a barrier function against permeation of the volatile substance. The barrier material can be, for example, polyamide, polyester, aluminum, or polyvinyl alcohol. The removable barrier layer is preferably embodied as a sleeve. Alternatively, it can be provided that the removable barrier layer is embodied as a release film.

In one apparatus according to one of the embodiments described in the foregoing having a first and a second and possibly a third housing part, it is preferably provided that the second housing part, starting from the closed position, is movable in the direction of the open position such that ambient air is suctioned into the housing and/or that the second housing part, starting from the open position, is movable in the direction of the closed position such that air enriched with the volatile substance is pressed out of the housing. Using successive opening and closing of the housing, i.e. successive transfer from the closed position to the open position and then back towards the closed position, it is possible to attain a brief stronger output of the volatile substance from the apparatus, for example, a brief stronger release of fragrance into the ambient air.

One alternative advantageous embodiment provides that the housing has a receiving element for the insert and one or a plurality of through-holes through which the volatile substance can be released into the ambient air, wherein the through-holes are provided with a removable barrier layer, for example a film. The barrier layer can be embodied, for example, as a release film. Alternatively, it can be provided that the removable barrier layer is a removable housing element made of the first material of the housing. For example, the removable housing element can be connected to the rest of the housing via a perforation. This perforation can be separated when the housing element is removed.

The insert (20) has a plurality of plys (21) connected to one another, in particular by an adhesive, in particular a plurality of plys of mechanical pulp board. The mechanical pulp board comprises a layer of ground wood pulp arranged between two cover strata. The cover strata each comprise pulp and/or paper. The plys (21) each have an area density in the range of 350 g/m² to 950 g/m², preferably 450 g/m² to 600 g/m², or 600 g/m² to 800 g/m².

Each of the plys (21) has a thickness in the range of 0.2 mm to 2.5 mm and an area density in the range of 350 g/m² to 950 g/m², preferably: a thickness in the range of 0.8 mm to 1.0 mm and an area density in the range of 350 g/m² to 400 g/m², or, a thickness in the range of 1.1 mm to 1.3 mm and an area density in the range of 450 g/m² to 550 g/m², or, a thickness in the range of 1.3 mm to 1.5 mm and an area density in the range of 490 g/m² to 570 g/m², or, a thickness in the range of 1.5 mm to 1.7 mm and an area density in the range of 580 g/m² to 660 g/m², or, a thickness in the range of 1.7 mm to 1.9 mm and an area density in the range of 630 g/m² to 750 g/m², or, a thickness in the range of 1.9 mm to 2.1 mm and an area density in the range of 700 g/m² to 850 g/m², or, a thickness in the range of 2.3 mm to 2.5 mm and an area density in the range of 800 g/m² to 1000 g/m².

The adhesive is biodegradable. The adhesive conforms to European Standard EN 13432 and is “completely biodegradable” according thereto. The insert (20) has a least a first ply (21), embodied in particular with the full surface area, and at least one second ply (21′) having at least one recess (22), in particular a punched hole. The second ply (21′) forms an outer surface of the insert (20). The insert (20) has a plurality of second plys (21′), wherein the recesses (22) of the plurality of second plys (21′) are arranged congruently above one another. A time-temperature indicator (17) is arranged on an outer side of the housing (10). The second housing part (14) have a basic outline of identical shape. A locking element that prevents complete separation of the two housing parts (13, 14) from one another is arranged on the first housing part (13) and/or second housing part (14). The first housing part (13) and the second housing part (14) are embodied identical. The third housing part has the shape of a tube.

A first locking element (16) is arranged on the third housing part (15) and cooperates with a further first locking element on the first housing part (13) and a second locking element (16) is arranged on the third housing part and cooperates with a further second locking element on the second housing part (14). A gap between the first housing part (13) and the second housing part (14) in the closed position is covered by a removable barrier layer having or embodied by a barrier material having a barrier function against permeation of the volatile substance.

The second housing part (14), starting from the closed position, is movable in the direction of the open position such that ambient air is suctioned into the housing (10) and/or in that the second housing part (14), starting from the open position, is movable in the direction of the closed position such that air enriched with the volatile substance is pressed out of the housing (10).

Further details, features, and advantages of these teachings result from the drawings and from the following description of preferred embodiments with the drawings. The drawings merely illustrate exemplary embodiments of these teachings that do not limit the inventive thought.

Further details, features, and advantages of the teachings herein result from the drawings and from the following description of preferred embodiments with the drawings. The drawings merely illustrate exemplary embodiments of the teachings herein that do not limit the inventive thought.

### BRIEF DESCRIPTION OF THE FIGURES

FIG. 1 is a perspective elevation of an apparatus for releasing a volatile substance into the ambient air according to a first exemplary embodiment in a closed position.

FIG. 2 illustrates the apparatus according to FIG. 1 when the vapor barrier layer is detached.

FIG. 3 illustrates the apparatus according to FIG. 1 in an open position.

FIG. 4 is a perspective elevation of an apparatus for releasing a volatile substance into the ambient air according to a second exemplary embodiment in a closed position.

FIG. 5 illustrates the apparatus according to FIG. 3 in an open position.

FIG. 6 is a perspective elevation of an apparatus for releasing a volatile substance into the ambient air according to a third exemplary embodiment in an open position.

FIG. 7 illustrates an inner housing part of a housing of the apparatus according to FIG. 6.

FIG. 8 illustrates the apparatus according to FIG. 6 in a closed position.

FIG. 9a is a top view of an insert of the apparatus according to FIG. 1.

FIG. 9b is a side view of an insert of the apparatus according to FIG. 1.

FIG. 10 a is a top view of an insert of the apparatus according to FIG. 4.

FIG. 10b is a side view of an insert of the apparatus according to FIG. 4.

FIG. 11 is a perspective elevation of an alternative insert of the apparatus according to FIGS. 1, 4, and 6.

### DETAILED DESCRIPTION

In the various figures, identical parts are always provided with the same reference number and therefore as a rule each is also identified or mentioned only once.

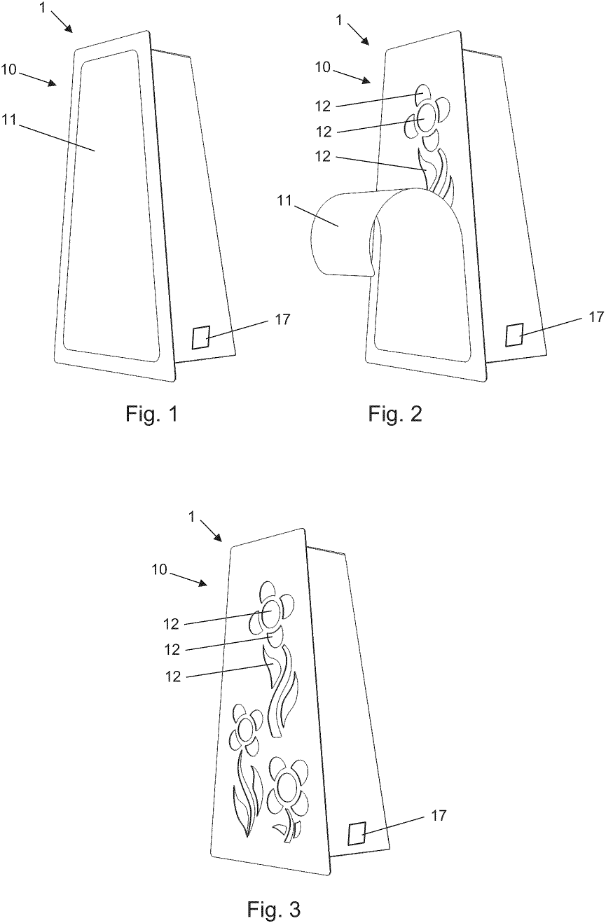

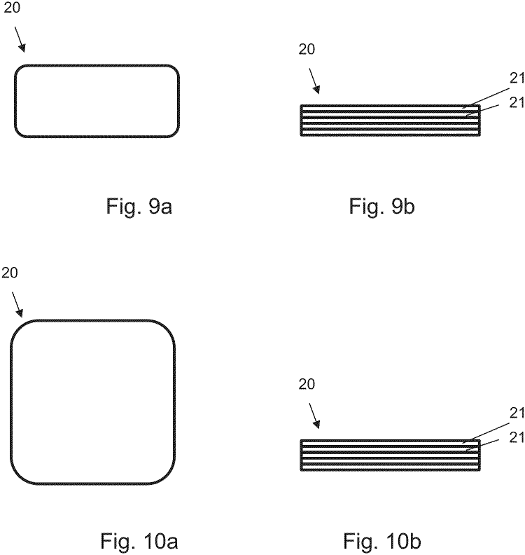

The depiction in FIG. 1 illustrates an apparatus 1 for releasing a volatile substance, for example, in the form of a fragrance or an insecticide, into the ambient air according to a first exemplary embodiment of the teachings herein. The apparatus 1 comprises a housing 10 made of a three-layer first material and further a barrier layer having a barrier function against permeation of the volatile substance as well as an insert 20, depicted in FIGS. 9a and 9b, arranged inside the housing 10. The insert 20 comprises a second material that is a plant-based fiber material, in particular paper, cardboard, or paperboard, wherein the second material is provided, in particular impregnated, with the volatile substance.

The housing 10 comprises a receiving element for the insert and one or a plurality of through-holes 12 through which the volatile substance can be released into the ambient air, wherein, in a closed position of the housing 10, the through-holes 12 are provided with a removable barrier layer 11, for example a film, see FIG. 1. Alternatively, it can be provided that the removable barrier layer 11 is a removable housing element made of the first material of the housing 10. For example, the removable housing element can be connected to the rest of the housing 10 via a perforation. This perforation can be separated when the housing element 11 is removed.

The depiction in FIG. 2 depicts by way of example removal of this barrier layer 11 from the through-holes 12.

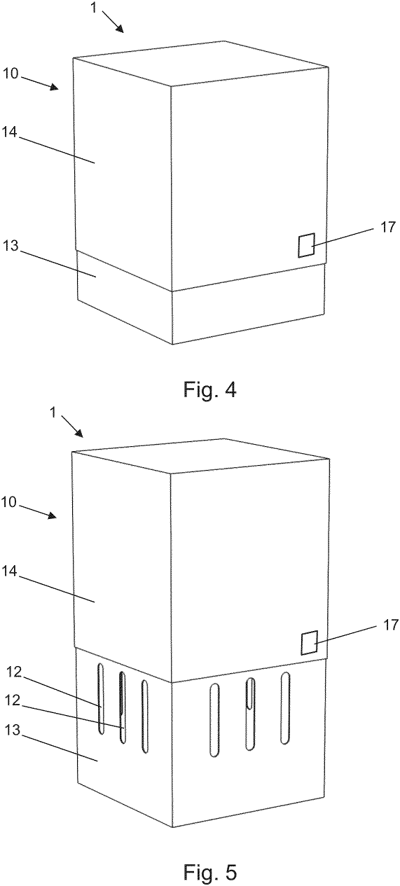

In FIG. 3, the housing 10 is shown in an open position in which the through-holes are exposed so that ambient air can circulate through the latter and the volatile substance, for example a fragrance, of the insert 20 can exit from the interior of the housing 10. The barrier layer 11 has been completely removed from the housing 10 according to FIG. 3.

The three-layer first material of the housing 10 of the apparatus 1 has a first layer made of paper, cardboard, or paperboard, a second layer made of polyethylene, and a third layer made of oriented polypropylene or biaxially oriented polyprobylene. It furthermore includes a barrier layer or barrier layer 11, for example comprising polyamide, polyester, aluminum, or polyvinyl alcohol. This barrier layer can be arranged in contact with a ply of the three-layer first material, in particular the paper, cardboard, or paperboard. Furthermore, it can be provided that the barrier layer is formed from that three-layer material made of paper, cardboard, or paperboard, polyethylene, and oriented polypropylene or biaxially oriented polypropylene.

Furthermore, a time-temperature indicator 17 via which the shelf life of the volatile substance can be displayed is arranged on the outside of the housing 10.

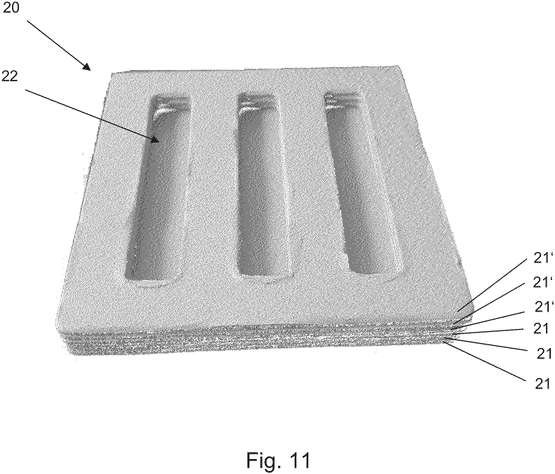

A second exemplary embodiment of an inventive apparatus 1 for releasing a volatile substance into the ambient air is depicted in FIGS. 4 and 5. The apparatus 1 according to the second exemplary embodiment also comprises a housing 10 made of a first three-layer material that is formed from a first layer made of paper, cardboard, or paperboard, a second layer made of polyethylene, and a third layer made of oriented polypropylene or biaxially oriented polypropylene. It furthermore has a further barrier layer having a barrier function against permeation of the volatile material, as well as an insert 20, depicted in FIGS. 10a and 10b, arranged inside the housing 10. The insert 20 comprises a second material that is a plant-based fiber material, in particular paper, cardboard, or paperboard, wherein the second material is provided, in particular impregnated, with the volatile substance.

What has been stated about the first material in accordance with the first exemplary embodiment also applies to the second exemplary embodiment. Therefore refer to the description of FIGS. 1-3.

In contrast to the first exemplary embodiment, the housing 10 according to the second exemplary embodiment comprises a first housing part 13 having a recess for the insert, wherein the first housing part 13 has one or a plurality of through-holes 12 through which the volatile substance can be released into the ambient air, and a second housing part 14, which can be displaced out of a closed position, shown in FIG. 4, in which position the second housing part covers the through-holes 12 of the first housing part, into an open position, shown in FIG. 5, in which the through-holes of the first housing part are exposed for releasing the volatile substance.

The housing 10 according to the second exemplary embodiment also has a time-temperature indicator 17 via which the shelf-life of the fragrance or insecticide can be displayed.

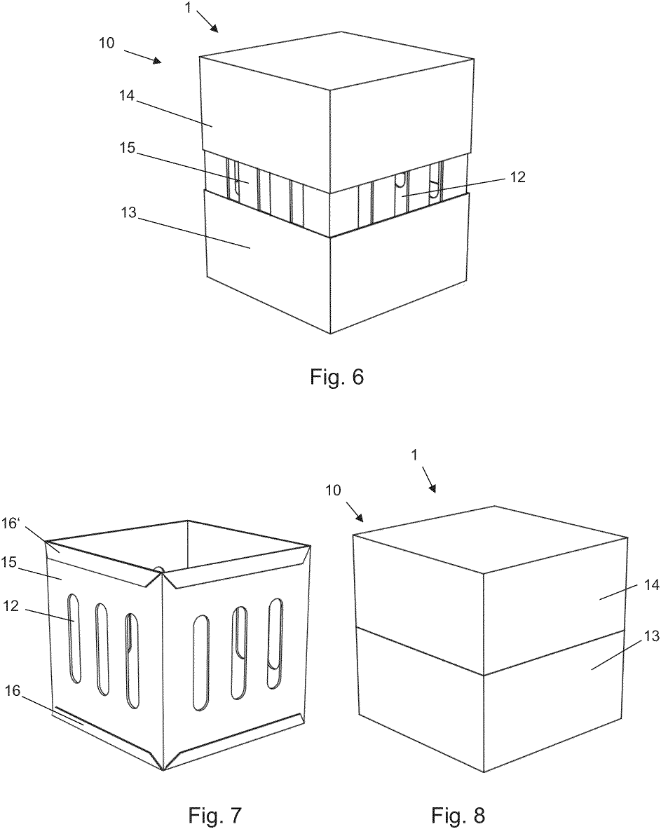

FIG. 6 depicts a third exemplary embodiment of an inventive apparatus 1 for releasing a volatile substance into the ambient air. The apparatus 1 according to the third exemplary embodiment also comprises a housing 10 made of a first three-layer material which is formed from a first layer made of paper, cardboard, or paperboard, a second layer made of polyethylene, and a third layer made of oriented polypropylene or biaxially oriented polypropylene, as well as a further barrier layer having a barrier function against permeation of the volatile substance, and an insert 20, depicted in FIGS. 10a and 10b and arranged inside the housing 10. The insert 20 comprises a second material that is a plant-based fiber material, in particular paper, cardboard, or paperboard, wherein the second material is provided, in particular impregnated, with the volatile substance.

What has been stated about the first material in accordance with the first exemplary embodiment also applies to the second exemplary embodiment. Refer to the description of FIGS. 1-3.

The housing 10 of the apparatus 1 according to the third exemplary embodiment comprises a first housing part 13, a second housing part 14, and a third housing part 15. The first and second housing parts 13, 14 are embodied identically in the exemplary embodiment. Both the first and also the second housing part 13, 14 have a receiving element for the insert 20; only one of the two housing parts 13, 14 is fitted with the insert 20, however.

The third housing part 15 is embodied like a tube or sleeve and comprises a plurality of through-holes 12 through which the volatile substance can be released into the ambient air if they are exposed by the first and/or second housing part 13, 14. In the closed position shown in FIG. 8, the first housing part 13 and the second housing part 14 cover the through-holes 12 of the third housing part 15. The first and second housing parts 13, 14 can be displaced from this closed position into an open position, shown in FIG. 6, in which the through-holes 12 of the third housing part 15 are exposed for releasing the volatile substance.

The housing 10 can additionally have a removable barrier layer, for example a barrier layer embodied as a sleeve, that covers a gap between the first housing part 13 and the second housing part 14 in the closed position. This barrier layer comprises a barrier material having a barrier function against permeation of the volatile substance or is embodied of such a material.

The third housing part 15 of the housing 10 is depicted in FIG. 7. It can be seen from this depiction that a plurality of first locking elements 16 are arranged on the third housing part and can cooperate with further first locking elements on the first housing part 13. Furthermore, the third housing part 15 comprises a plurality of second locking elements 16′ that can cooperate with further second locking elements on the second housing part 14. The first locking elements 16 are arranged on a first side of the third housing part 15 opposing a second side of the third housing part on which the second locking elements 16′ are arranged. Thus, when the housing is assembled, the first locking elements 16 face the first housing part 13 and the second locking elements 16′ face the second housing part. The first and second locking elements 16, 16′ are embodied as folded segments of the third housing part 13.

FIG. 8 depicts a housing 10 in the closed position having a first housing part 13 and a second housing part 14.

FIGS. 9a and 9b depict the insert 20 of the first exemplary embodiment. This insert 20 has a rectangular section with rounded corners. The insert 20 comprises a plurality of, in this case five, plys 21 of the plant-based fiber material, for example mechanical pulp board or wood pulp, connected to one another by an adhesive. The adhesive is preferably biodegradable. The plys each have an area density in the range of 350 g/m² to 950 g/m², preferably 450 g/m² to 600 g/m², or 600 g/m² to 800 g/m². Alternatively it can be provided that the plys each have a thickness in the range of 0.8 mm to 2.5 mm and an area density in the range of 350 g/m² to 950 g/m².

FIGS. 10a and 10b depict the insert 20 of the second and third exemplary embodiments. This insert 20 has a square section with rounded corners. The insert 20 comprises a plurality of, in this case five, plys 21 of the plant-based fiber material, for example mechanical pulp board or wood pulp, connected to one another by an adhesive. The adhesive is preferably biodegradable. The plys each have an area density in the range of 350 g/m² to 950 g/m², preferably 450 g/m² to 600 g/m², or 600 g/m² to 800 g/m². Alternatively, it can be provided that the plys each have a thickness in the range of 0.8 mm to 2.5 mm and an area density in the range of 350 g/m² to 950 g/m².

The exemplary embodiments of the inserts 20 depicted in FIGS. 6a, 6b, 7a, and 7b are merely by way of example and can have a different shape, e.g. can be pentagonal, hexagonal, or octagonal, and/or can have sharp, non-rounded corners.

FIG. 11 depicts an insert 20 that can be used as alternative in the exemplary embodiments described in the foregoing. The insert 20 comprises a plurality of, in this case six, plys 21 of the plant-based fiber material, for example mechanical pulp board or wood pulp, connected to one another by an adhesive. The insert 20 as a plurality of, in this case three, first plys 21 that are embodied with the full surface area. The insert 20 furthermore has a plurality of, in this case three, second plys 21′ that include a plurality of, in this case three, in particular punched, holes. One of the second plys 21′ forms an outer surface of the insert 20. The recesses 22 of the second plys 21′ are arranged congruently above one another and form a receiving element for volatile material.

The inserts 20 described in the foregoing can alternatively comprise one of the following materials or one or a plurality of the cited materials: a further plant-based fiber material or sucrose or starch or a mineral material. The plant-based fiber material can be mechanical pulp, in particular ground wood pulp, or pulp or cotton fiber or grass fiber. The mineral material can be rock flour or bentonite or silica gel.

List of reference numbers

| | |
|---|---|
| 1 | Apparatus for releasing a fragrance or insecticide into the ambient air |
| 10 | Housing |
| 11 | Barrier layer |
| 12 | Through-hole |
| 13 | First housing part |
| 14 | Second housing part |
| 15 | Third housing part |
| 16, 16′ | Locking element |
| 17 | Time-temperature indicator |
| 20 | Insert |
| 21, 21′ | Ply |
| 22 | Recess |

1. An apparatus for releasing a volatile substance into the ambient air, comprising:
a) a housing that contains an at least three-layer first material, wherein
(i) a first layer of the at least three-layer first material contains or comprises paper or cardboard or paperboard,
(ii) a second layer of the at least three-layer first material contains or comprises polyethylene, and
(iii) a third layer of the at least three-layer first material contains or comprises oriented polypropylene, the third layer has a barrier function against permeation of the volatile substance, and
(iv) optionally at least one further barrier layer having a barrier function against permeation of the volatile substance,
b) an insert arranged inside the housing and made of a second material, the second material is provided or impregnated, with the volatile substance, the second material is one of the following materials or has one or a plurality of the specified materials: i) a plant-based fiber material or ii) sucrose or starch or iii) a mineral material.

2. The apparatus according to claim 1, wherein the polyethylene includes or is low-density polyethylene (LDPE/PE-LD), low linear density polyethylene (LLDPE/PE-LLD), and/or high density polyethylene (HDPE/PE-HD).

3. The apparatus according to claim 1, wherein the oriented polypropylene includes or is OPP (oriented polypropylene) or BOPP (biaxially oriented polypropylene).

4. The apparatus according to claim 1, wherein a further barrier layer comprises polyamide, polyester, aluminum, or polyvinyl alcohol.

5. The apparatus according to claim 1, wherein the first layer and/or the second layer and/or the third layer has/have at least in part an acrylic coating.

6. The apparatus according to claim 1, wherein the third layer is water-repellant and/or transparent.

7. The apparatus according to claim 1, wherein the at least three-layer first material comprises:
(i) a first layer made of paper, cardboard, or paperboard,
(ii) a second layer made of polyethylene,
(iii) a third layer made of polypropylene, wherein this layer
has a barrier function against permeation of the volatile substance, and optionally, at least one further barrier layer having a barrier function against permeation of the volatile substance.

8. The apparatus according to claim 1, wherein the housing has a stand surface on which the housing can stand on a subsurface, wherein the stand surface is water-repellant.

9. The apparatus according to claim 1, wherein the plant-based fiber material is paper or cardboard or paperboard, or mechanical pulp board, or wood pulp.

10. The apparatus according to claim 1, wherein the plant-based fiber material of the second material is mechanical pulp, ground wood pulp, or pulp or cotton fiber or grass fiber.

11. The apparatus according to claim 1, wherein the mineral material is rock flour or bentonite or silica gel.

12. The apparatus according to claim 1, wherein the insert has a plurality of plys of the second material connected to one another by an adhesive.

13. The apparatus according to claim 1, wherein the housing has a first housing part and a second housing part, the first housing part and/or the second housing part comprises a receiving element for the insert, wherein the first housing part has one or a plurality of through-holes through which the volatile substance can be released into the ambient air, and the second housing part can be displaced from a closed position, in which it covers the through-holes of the first housing part, to an open position, in which the through-holes of the first housing part are exposed for releasing the volatile substance.

14. The apparatus according to claim 12, wherein the housing has a first housing part, a second housing part, and a third housing part, the first housing part and/or the second housing part comprises a receiving element for the insert, the third housing part has one or a plurality of through-holes through which the volatile substance can be released into the ambient air, wherein the first housing part and/or the second housing part can be displaced from a closed position, in which the first housing part and/or the second housing part covers the through-holes of the third housing part, to an open position, in which the through-holes of the third housing part are exposed for releasing the volatile substance.

15. Apparatus according to claim 13, wherein the housing has a recess for the insert and one or a plurality of through-holes through which the volatile substance can be released into the ambient air, wherein the through-holes are provided with a removable barrier layer or a film.
